(19) United States
(12) Patent Application Publication
(10) Pub. No.: US 2026/0166111 A1
(43) Pub. Date: Jun. 18, 2026

(54) Methods of treatment involving tobacco leaf extracts to help smoking cessation

(71) Applicant: NFL BIOSCIENCES, Castelnau-le-Lez (FR)

(72) Inventor: Bruno LAFONT, MONOBLET (FR)

(21) Appl. No.: 19/123,596

(22) PCT Filed: Oct. 23, 2023
(86) PCT No.: PCT/IB2023/000626

§ 371 Date: Apr. 23, 2025

Related U.S. Application Data
(60) Provisional application No. 63/418,729, filed on Oct. 24, 2022.

(51) Int. Cl. A61K 36/81; A61K 9/00; A61K 9/08; A61K 31/465; A61P 25/34

(52) U.S. Cl. CPC A61K 36/81; A61K 9/0019; A61K 9/08; A61K 31/465; A61P 25/34; A61K 2236/331

(57) ABSTRACT

The disclosure generally relates to methods of treatment to help a subject cess smoking, comprising at least one step during which the subject is administered an effective dose of an aqueous solution of an enriched protein tobacco leaf extract during which the subject continues to smoke ad libitum, and optionally a second step during which the subject is provided with one or several optional complementary administrations of at least one product capable of controlling the symptoms of nicotine withdrawal, such as a nicotine agonist or nicotine partial agonist, and/or a nicotine substitute. The disclosure also relates to a kit comprising an enriched protein tobacco leaf extract and at least one product capable of controlling the symptoms of nicotine withdrawal of nicotine, such as a nicotine agonist or nicotine partial agonist and/or a nicotine substitute.

### BACKGROUND

The disclosure generally relates to methods of treatment to help a subject cess smoking, comprising at least one step during which the subject is administered an effective dose of an aqueous solution of an enriched protein tobacco leaf extract during which the subject continues to smoke ad libitum, and optionally a second step during which the subject is provided with one or several optional complementary administrations of at least one product capable of controlling the symptoms of nicotine withdrawal, such as a nicotine agonist or nicotine partial agonist, and/or a nicotine substitute. The disclosure also relates to a kit comprising an enriched protein tobacco leaf extract and at least one product capable of controlling the symptoms of nicotine withdrawal of nicotine, such as a nicotine agonist or nicotine partial agonist and/or a nicotine substitute.

Tobacco addiction is one of the biggest public health threats the world has ever faced, killing nearly 8 million people a year. There are more than one billion smokers in the world, up to half of them will eventually die of a tobacco-related disease. Approximately one person dies every six seconds due to tobacco, accounting for one in ten adult deaths. Tobacco caused one hundred million deaths in the 20th century. If current trends continue, it may cause one billion deaths in the 21st century.

As per National Tobacco Control Programme of India, the tobacco epidemic is one of the major public health threats with one million deaths attributable to tobacco each year in India.

As per Global Adult Tobacco Survey (GATS-2), in India, every tenth adult (10.0 crore) smokes tobacco and 28.6% of adults aged 15 and above (26.7 crore) use tobacco in any form; 19.9 crore adults in rural areas and 6.8 crore adults in urban areas use tobacco.

There are more than 4000 chemicals in tobacco smoke, of which at least 250 are known to be harmful and more than 50 are known to cause cancer. Tobacco is a risk factor for six of the eight leading causes of death. It can cause cancer almost anywhere in the body. In industrialized countries, it is estimated to cause over 90% of lung cancer in men and about 70% of lung cancer among women, and about 22% of all cardiovascular disease. About 90% of all deaths from chronic obstructive pulmonary disease are caused by smoking. It harms nearly every organ of the body and affects a person's overall health.

Specifically in the smoking addiction, the assumption is that nicotine is involved in the tobacco dependency mechanism. Nicotine has thus been considered up to today as the principal reinforcing psychoactive and addictive substance. Nicotine can indeed bind to proteins present at the surface of the nerve cells, the nicotinic acetylcholine receptors. In the presence of nicotine, these receptors, which are in fact channels, open. A cascade of events then follows, resulting in the release of a hormone, dopamine. The nicotine stimulates the “reward circuit” and thus produces a sensation of satisfaction. Therefore, when people stop smoking, their body “craves” its dose of nicotine in order to satisfy this sensation of wellbeing: these are the symptoms of nicotine withdrawal. The purpose of the nicotine substitutes for example, is therefore to provide the smoker's brain with a quantity of nicotine sufficient to prevent withdrawal symptoms. To summarize, nicotine targets specific brain receptors resulting in the release of neurotransmitters such as dopamine in the central nervous system that generate positive sensations, such as pleasure, relaxation and appetite suppression. Over time the receptors become conditioned to expect nicotine, and when deprived, the smoker experiences its withdrawal symptoms.

Smoking decreases also the levels of monoamine oxidase (MAO), an enzyme responsible for the breakdown of dopamine, and results in higher dopamine levels and tobacco dependence. As nicotine itself does not interact with MAO, this change is likely due to other tobacco smoke ingredient(s) and is another cause of tobacco addiction. These ingredients also disrupt the coupling between noradrenergic and serotonergic systems through the desensitization of 5-HT(1A) auto receptors, increasing the reactivity of dopaminergic neurons. A recent study showed that medial prefrontal cortex (mPFC) glutamatergic and gamma-aminobutyric acid (GABA)ergic receptors may be also involved, through a GABAergic plasticity mechanism in the dorsal mPFC. Several other publications mention a plurality of mechanisms involved in tobacco addiction. Smoking behavior also varies with genetic and environmental factors. Tobacco addiction mechanism is then highly complex, involves several pathways in the CNS and other molecules in addition to nicotine.

Dependence or addiction has been defined by the World Health Organization as “a syndrome for which the consumption of a product becomes a requirement greater than those of other behaviours which were previously more important. In its extreme form the state of dependence is characterized by an irresistible need for a product which compels the individual suffering from this dependence to impulsively seek this product”.

For all these reasons and/or for others, many smokers therefore seek quitting smoking.

Smokers can choose to quit abruptly by setting a quit day to stop smoking in one step (abrupt quitting) or to reduce their consumption before the quit day (gradual quitting). In the case of gradual quitting, a ten day to 2-week reduction phase before the quit day is recommended in order to not alter motivation.

The first two weeks following the target quit date (TQD) are significantly correlated to a 6-month abstinence. Smokers who do not relapse after the TQD increase their odds of becoming long term abstinent.

Current medications for smoking cessation include Nicotine Replacement Therapies (NRTs), Nicotine free quit medication which are bupropion hydrochloride (ZYBAN®) and varenicline tartrate (CHAMPIX® in France or CHANTIX® in the USA). Several nicotine vaccines have been under development.

Nicotine Replacement Treatments were the first pharmacological treatments approved for use in smoking cessation therapy. They are usually used in conjunction with behavioral support. They include nicotine gums, transdermal patches, tablets, nasal sprays, inhalation systems which act as nicotine substitute to reduce withdrawal symptoms and urge to smoke. A meta-analysis from 117 trials from the Cochrane Tobacco Addiction Group trials in 2012 showed that all commercialized NRT can help people to increase their chances of successfully stopping smoking by 50-70% at 6 months. Nevertheless, the absolute abstinence rates for these medications are modest, and most smokers relapse: 6-month quit rates among smokers who use NRT are 20-30%, and 70-80% of smokers who use these products relapse. NRT can be used in both abrupt and gradual quitting. In abrupt quitting NRT are used during eight to twelve weeks from the TQD. In gradual quitting they can be used during six weeks to six months before the TQD to help smokers reducing their consumption and during eight to twelve weeks from the TQD.

ZYBAN® (bupropion hydrochloride) commercialized by GlaxoSmithKline was the first nicotine free quit medication approved in 1997. According to French guidelines, Bupropion is superior to placebo for smoking cessation at six months, but is not superior to NRT, and is less superior than varenicline. It has a high level of side effects: risk of depression and/auto-aggressive suicidal behaviour, or allergic skin reactions, neuropsychiatric and neurological disorders, and cardiovascular effects including hypertension, angina and/or myocardial infarction, dry mouth, nausea. Bupropion is recommended to use as last choice to treat particular cases with close monitoring of subjects.

CHAMPIX® (Varenicline tartrate) commercialized by Pfizer was approved in 2006. Varenicline is a selective partial agonist for α4β2 nicotinic acetylcholine receptor subtype and blocks the ability of nicotine to stimulate the central nervous mesolimbic dopamine system. Pfizer assessed CHAMPIX® efficacy in six clinical trials including a total of 3659 chronic cigarette smokers. The maximal performance of CHAMPIX® obtained over a year was below 30% except when the treatment was maintained for six months (>50%). A meta-analysis by Cahill et al. in 2013 showed a superiority of varenicline over placebo, bupropion and NRTs, but not over a combination of NRTs (transdermal patch+oral NRT). This product is associated with serious side effects such as neuropsychiatric symptoms including depression and suicidal/self-injurious behaviour, behavioural changes, hostility, agitation. It has also been described as associated with sleep and gastrointestinal disorders and cardiovascular troubles, but these last observations are yet to be confirmed. Because of these adverse effects, especially in relation to suicide and depression, it is recommended as second-line after failure of nicotine replacement therapy.

Vaccine therapy was also under evaluation. Nicotine is the target. Nicotine molecules are bound chemically to a carrier protein or particle in order to stimulate the immune system to produce antibodies that bind to nicotine from smoke and prevent it from entering the brain. NIC002 Cytos/Novartis) and NicVAX® (Nabi/GSK) vaccines failed to show efficacy in respectively phase II and phase III clinical trials. Nic7 was a bioconjugate vaccine developed by Pfizer evaluated in 200 individuals in a phase 1 clinical trial and stopped in 2016. SEL-068 (Selecta Biosciences, Inc.) is a nanoparticle-based vaccine in phase I of clinical development in 2012-2014, but no results or recent developments have been published.

Several other products are under evaluation for smoking cessation, including, BP1.4979, a D3R partial agonist (phase II, Bioprojet), nadolol (phase II, Invion, Inc.), Moclobemide, a Reversible MAO-A Inhibitor (phase II, Duke University Medical Center/Philip Morris), and EVP-6124, an Alpha-7 Nicotinic Acetylcholine Receptor Agonist (ForumPharmaceuticals Inc).

It is to be also mentioned that electronic cigarettes are gaining popularity but are not approved for smoking cessation on many regulatory jurisdictions. The development of safe and efficient alternatives for this indication is more than ever called for.

As described in WO2017174787, the present inventor found that the injection of an aqueous solution of an aqueous extract of tobacco leaves makes it possible to reduce dependence. More particularly, it was possible to demonstrate that a single injection of a tobacco extract according to WO2017174787 was generally sufficient to reduce or even eliminate smokers' tobacco dependence. This feature constitutes a major advantage for the subject, given that the products currently available on the market only provide treatments which are long-term and in several doses. Conversely, the treatment according to the invention proposes a shock therapy, i.e., a treatment constituted preferably of a single injection of a tobacco extract, optionally followed after several days or even weeks by at least one second injection if the treated subject feels the need.

The inventor has now discovered that his previously found solution, when used under certain circumstances, in particular with other products capable of controlling the symptoms of nicotine withdrawal, enables an easier weaving off cigarette addiction.

### BRIEF SUMMARY

The disclosed invention concerns a method to precondition a subject to cess smoking comprising a step wherein the subject is administered percutaneously or subcutaneously an effective dose of an aqueous solution of an enriched protein tobacco leaf extract, essentially devoid of nicotine, during which the subject continues to smoke ad libitum.

Such a method advantageously enables the subject to more easily stop smoking in comparison to existing methods. Indeed, the preconditioning of the subject eases him to more effectively quit smoking because of the sense of distaste or even disgust generated by the administration of the aqueous solution of an enriched protein tobacco leaf extract before the subject actually stops smoking.

The disclosed invention thus also concerns a method of treatment of smoking cessation of a subject, comprising the following two successive steps:
i) a preconditioning step during which the subject is administered percutaneously or subcutaneously at least one effective dose of an aqueous solution of an enriched protein tobacco leaf extract, essentially devoid of nicotine, during which the subject continues to smoke ad libitum, and
ii) a second step following the preconditioning step i), within 2 to 180 days, preferably within 2 to 30 days, from said administration to the subject carried out during the preconditioning step i) (also called “initial step i)”), during which the subject completely quits smoking with one or several optional complementary administrations of at least one product capable of controlling the symptoms of nicotine withdrawal.

By “product capable of controlling the symptoms of nicotine withdrawal”, it is meant in the scope of the present invention products which are not an aqueous solution of an enriched protein tobacco leaf extract as found in step i). For example, such a product may be an agonist of nicotine or nicotine itself which is administered as known in the art, for example in a nicotine patch or gum or other.

The preconditioning phase (i.e. step (i) in the method of treatment and the method to precondition a subject to cess smoking) with an aqueous solution of an enriched protein tobacco leaf extract, essentially devoid of nicotine, means that the day the subject decides to stop smoking, that is to say the day when he/she will no longer have any nicotine intake from his/her cigarettes, this intake can be substituted if necessary by at least one product capable of controlling the symptoms of nicotine withdrawal. The weaning off cigarettes is thus eased.

The second step (ii) helps the subject to definitely quit smoking, in particular by weaning off the nicotine addiction.

The disclosed invention also concerns a kit comprising:
an aqueous solution of an enriched protein tobacco leaf extract; and separately
at least one product capable of controlling the symptoms of nicotine withdrawal.

Such a kit may help the subject apply the method of treatment according to the present invention by providing the necessary products, potential instructions and/or safety measures to use them.

### DETAILED DESCRIPTION

While the presently disclosed inventive concept(s) is susceptible of various modifications and alternative constructions, certain illustrated embodiments thereof are described below in details.

### Methods

It was found that a subject could be preconditioned to cess smoking by administering percutaneously or subcutaneously to said subject an effective dose of an aqueous solution of an enriched protein tobacco leaf extract, essentially devoid of nicotine, during which the subject continues to smoke ad libitum.

To reinforce the action of the method, if need-be, the subject may be administered percutaneously or subcutaneously a second effective dose of an aqueous solution of an enriched protein tobacco leaf extract, essentially devoid of nicotine, during which the subject continues to smoke ad libitum. Although, it was found that such a second administration is usually not needed, to counter any odds, it may be advised to readminister an effective dose of an aqueous solution of an enriched protein tobacco leaf extract, if the subject does not experience the distaste or disgust intended by the first administration of an aqueous solution of an enriched protein tobacco leaf extract.

A way to help the subject determine if he/she needs a second effective dose of an aqueous solution of an enriched protein tobacco leaf extract, essentially devoid of nicotine, may be provided by giving a score questionnaire wherein the questions asked enable a subjective evaluation of the need and/or pleasure to smoke. Such a questionnaire may be provided by asking e.g., one or several of the following questions (or similar ones): (1) “Was smoking satisfying?”, (2) “Did cigarettes taste good?”, (3) “Did you enjoy the sensations in your throat and chest?”, (4) “Did smoking calm you down?”, (5) “Did smoking make you feel more awake?”, (6) “Did smoking make you feel less irritable?”, (7) “Did smoking help you concentrate?”, (8) “Did smoking reduce your hunger for food?”, (9) “Did smoking make you dizzy?”, (10) “Did smoking make you nauseous?”, (11) “Did smoking immediately relieve your craving for a cigarette?”, (12) “Did you enjoy smoking?”.

For example, each question may be answered by giving a score from 1 to 7, depending on the feeling of the subject, whether each question is not at all fulfilled (score equals 1) or totally fulfilled (score equals to 7), with a middle score of 4 out of 7.

The questionnaire may comprise 1, 2, 3, 4, 5, 6, 7, 8, 9, 10, 11 or all 12 of the here-above questions.

In one embodiment, this questionnaire comprises all twelve questions listed here-above and is so-called a “Modified Cigarette Evaluation Questionnaire (mCEQ)”. The modified Cigarette Evaluation Questionnaire (mCEQ) uses three multi-item subscales and two single items: “Smoking Satisfaction” (items 1, 2, and 12); “Psychological Reward” (items 4 through 8); “Aversion” (items 9 and 10); “Enjoyment of Respiratory Tract Sensations” (item 3); and “Craving Reduction” (item 11). Scores for each subscale are calculated as the mean of the individual item responses or the single item. Higher scores indicate greater intensity on that scale.

Preferably, the second effective dose of an aqueous solution of an enriched protein tobacco leaf extract, essentially devoid of nicotine, may be administered within 2 to 15 days, preferably within 5 days, from its first administration.

Preferably, the present invention concerns a method of preconditioning a subject to stop smoking, wherein said extract of tobacco leaves contains at least 5% by weight, based on the total weight of a dry extract thereof, of proteins of molecular mass greater than 10 kDa, and is essentially free of molecules of molecular mass less than 10 kDa and of RuBisCO proteins; and

wherein the content, in the extract of tobacco leaves, of proteins whose molecular mass is greater than 500 kDa, is less than 15% by weight, based on the total protein weight of the dry extract.

In an embodiment, the present invention concerns a method of preconditioning a subject to stop smoking, wherein the proteins are selected from the group consisting of the following protein families: lignin-forming anionic peroxidase, glucan endo-1,3-beta-glucosidase, endochitinase, pathogenesis-related protein, osmotin, proteinase inhibitors, and mixtures thereof.

Preferably, the present invention concerns a method of preconditioning a subject to stop smoking, wherein the content, in the extract of tobacco leaves, of proteins whose molecular mass is greater than 100 kDa is less than 15% by weight based on the total protein weight of the dry extract.

Preferably, the present invention concerns a method of preconditioning a subject to stop smoking, wherein the extract of tobacco leaves comprises at least one protein belonging to the family of glucan endo-1,3-beta-glucosidases selected from the group consisting of beta-1,3-endoglucanase acidic isoform PR-Q′ (PR36401 according to the UniProt database), beta-1,3-endoglucanase basic vacuolar isoform GLB (P27666 according to the UniProt database), and mixtures thereof.

Preferably, the present invention concerns a method of preconditioning a subject to stop smoking, wherein the extract of tobacco leaves comprises at least one protein belonging to the family of endochitinases selected from the group consisting of acidic endochitinase P (P17513 according to the UniProt database), acidic endochitinase Q (P17514 according to the UniProt database), endochitinase B (P24091 according to the UniProt database), and mixtures thereof.

Preferably, the present invention concerns a method of preconditioning a subject to stop smoking, wherein the extract of tobacco leaves comprises at least osmotin (P14170 according to the UniProt database).

Preferably, the present invention concerns a method of preconditioning a subject to stop smoking, wherein the extract of tobacco leaves comprises at least one lignin-forming anionic peroxidase (P11965 according to the UniProt database).

Preferably, the present invention concerns a method of preconditioning a subject to stop smoking, wherein the extract of tobacco leaves comprises at least one pathogenesis-related protein selected from the group consisting of-pathogenesis-related protein R (P13046 according to the UniProt database), pathogenesis-related protein PR-4A (PR29062 according to the UniProt database), pathogenesis-related protein PR-4B (PR29063 according to the UniProt database), and mixtures thereof.

Preferably, the present invention concerns a method of preconditioning a subject to stop smoking, wherein the extract of tobacco leaves comprises at least one protein belonging to the family of proteinase inhibitors selected from the group consisting of proteinase inhibitor I-B (Q03199 according to the UniProt database), proteinase inhibitor I-A (Q03198 according to the UniProt database), and mixtures thereof.

Preferably, the present invention concerns a method of preconditioning a subject to stop smoking, wherein the protein content in the dry extract of the extract of tobacco leaves is at least 10% by weight based on the total weight of the dry extract.

Preferably, the present invention concerns a method of preconditioning a subject to stop smoking, wherein the proteins present in the extract of tobacco leaves are present in an amount ranging from 1 to 1 000 μg/m L.

Preferably, the present invention concerns a method of preconditioning a subject to stop smoking, wherein the extract of tobacco leaves or the composition is provided in a form suitable for administration by subcutaneous injection, in a form suitable for administration by means of an adhesive transdermal therapeutic system such as a patch, or in a form suitable for administration by spraying or by vaporization.

Preferably, the present invention concerns a method of preconditioning a subject to stop smoking, wherein the extract of tobacco leaves or the composition is provided in a dosage form of 0.03 mL to 10 mL.

As disclosed above another aspect of the present disclosure concerns a method of treatment of smoking cessation of a subject, comprising the following two successive steps:
i) a preconditioning step during which the subject is administered percutaneously or subcutaneously at least one effective dose of an aqueous solution of an enriched protein tobacco leaf extract, essentially devoid of nicotine, during which the subject continues to smoke ad libitum, and
ii) a second step following the preconditioning step i), within 2 to 180 days, preferably within 2 to 30 days, from said administration to the subject carried out during the preconditioning step i), during which the subject completely quits smoking with one or several optional complementary administrations of at least one product capable of controlling the symptoms of nicotine withdrawal.

As mentioned above, the aim of the administration of an effective dose of an aqueous solution of an enriched protein tobacco leaf extract, essentially devoid of nicotine, is to provoke a distaste or disgust of cigarettes. However, also as mentioned above, cigarettes contain numerous addictive components, specifically nicotine. Therefore, should the subject encounter one or several side effects linked to nicotine withdrawal, the administration of a product capable of controlling the symptoms of nicotine withdrawal, such as e.g., a nicotine patch, will help the subject to overcome nicotine addiction specifically. A way to help the subject determine if he/she needs such substitutive treatment regarding nicotine, may be provided by giving a score questionnaire with known symptoms of nicotine withdrawal effects.

The following adverse events may occur due to nicotine withdrawal: depression, insomnia, irritability/frustration/anger, anxiety, difficulty concentrating, restlessness, increased appetite/weight gain, constipation, bradycardia ...

For example, questions such as “have you noticed in the last two days, four days, etc. a mood change?”, “have people around you commented that you are in a bad mood since you stopped smoking?” or “have you noticed in the last two days, four days, etc being more hungry than usual”, etc. with a score chart to help the subject determine his need in nicotine.

A way to help the subject determine if he/she may pass to step (ii) in the here-above method to completely quit smoking, may be provided by giving a score questionnaire as disclosed above wherein the questions asked, enable a subjective evaluation of the need and/or pleasure to smoke. Such a questionnaire may be provided by asking e.g., one or several of the following questions (or similar ones): (1) “Was smoking satisfying?”, (2) “Did cigarettes taste good?”, (3) “Did you enjoy the sensations in your throat and chest?”, (4) “Did smoking calm you down?”, (5) “Did smoking make you feel more awake?”, (6) “Did smoking make you feel less irritable?”, (7) “Did smoking help you concentrate?”, (8) “Did smoking reduce your hunger for food?”, (9) “Did smoking make you dizzy?”, (10) “Did smoking make you nauseous?”, (11) “Did smoking immediately relieve your craving for a cigarette?”, (12) “Did you enjoy smoking?”.

For example, each question may be answered by giving a score from 1 to 7, depending on the feeling of the subject, whether each question is not at all fulfilled (score equals 1) or totally fulfilled (score equals to 7), with a middle score of 4 out of 7.

The questionnaire may comprise 1, 2, 3, 4, 5, 6, 7, 8, 9, 10, 11 or all 12 of the here-above questions.

In one embodiment, this questionnaire comprises all twelve questions listed here-above and is so-called a “Modified Cigarette Evaluation Questionnaire (mCEQ)”. The modified Cigarette Evaluation Questionnaire (mCEQ) uses three multi-item subscales and two single items: “Smoking Satisfaction” (items 1, 2, and 12); “Psychological Reward” (items 4 through 8); “Aversion” (items 9 and 10); “Enjoyment of Respiratory Tract Sensations” (item 3); and “Craving Reduction” (item 11). Scores for each subscale are calculated as the mean of the individual item responses or the single item. Higher scores indicate greater intensity on that scale.

This questionnaire may be answered before, during and/or after step (i) and the results may be compared to each other's to monitor the effect(s) of the administrations made and thus adapt the treatment to be made (e.g., by adding more and/or another product capable of controlling the symptoms of nicotine withdrawal).

In an embodiment, the present invention concerns a method of treatment, wherein the preconditioning step comprises two or three percutaneous administrations of effective doses of two or three aqueous solutions of an enriched protein tobacco leaf extract, essentially devoid of nicotine, during which the subject continues to smoke ad libitum.

Preferably, the present invention concerns a method of treatment, wherein the two or three percutaneous administrations of effective doses of two or three aqueous solutions of an enriched protein tobacco leaf extract, essentially devoid of nicotine, during which the subject continues to smoke ad libitum, are separated one from each other by at least 10 days, preferably at least 20 days.

Preferably, the present invention concerns a method of treatment, wherein the preconditioning step consists of one percutaneous administration of an effective dose of an aqueous solution of an enriched protein tobacco leaf extract, essentially devoid of nicotine, during which the subject continues to smoke ad libitum.

Preferably, the present invention concerns a method of treatment, wherein the second step following the preconditioning step i) is within 2 to 120 days, preferably within 2 to 60 days, from said administration to the subject carried out during the preconditioning step i), during which the subject completely quits smoking with one or several optional complementary administrations of at least one product capable of controlling the symptoms of nicotine withdrawal.

More preferably, the present invention concerns a method of treatment, wherein the second step following the preconditioning step i) is within 2 to 30 days from said administration to the subject carried out during the preconditioning step i), during which the subject completely quits smoking with one or several optional complementary administrations of at least one product capable of controlling the symptoms of nicotine withdrawal.

In an embodiment, the present invention concerns a method of treatment, wherein said one or several optional complementary administrations of at least one product capable of controlling the symptoms of nicotine withdrawal is conducted in a way of an accompaniment of the subject to gradually wean the subject off nicotine. This enables to control on a period of time the compensation of nicotine withdrawal. Preferably, the accompaniment is characterized in that the subject is consulted every month, preferably every fortnight, every week, every 6 days, every 5 days, every 4 days, every 3 days, every 2 days or even every day on a period of time ranging between one week and 12 months, preferably between two weeks and 6 months, more preferably between 1 month and 4 months such as 3 months.

In an embodiment, the present invention concerns a method of treatment, wherein when several products capable of controlling the symptoms of nicotine withdrawal are administered, they are administered independently in time one from each other. This enables a more precise and adaptable treatment of the smoking cessation, in particular of the weaning off of the nicotine.

Preferably, the present invention concerns a method of treatment, wherein said at least one product capable of controlling the symptoms of nicotine withdrawal is chosen from a nicotine agonist or partial agonist, a nicotine substitutes, or a combination thereof.

Preferably, the present invention concerns a method of treatment, wherein said nicotine agonist or partial agonist is chosen from varenicline, cytisine, lobeline, or a combination thereof.

In an embodiment, the present invention concerns a method of treatment, wherein the second step is conducted within 3 to 10 days from said administration to the subject carried out during the preconditioning step i).

Preferably, the present invention concerns a method of treatment, wherein the second step is conducted within 3 to 4 days from said administration to the subject carried out during the preconditioning step i).

In an embodiment, the present invention concerns a method of treatment, wherein the extract of tobacco leaves contains at least 5% by weight, based on the total weight of a dry extract thereof, of proteins of molecular mass greater than 10 kDa, and is essentially free of molecules of molecular mass less than 10 kDa and of RuBisCO proteins; and

wherein the content, in the extract of tobacco leaves, of proteins whose molecular mass is greater than 500 kDa, is less than 15% by weight, based on the total protein weight of the dry extract.

Preferably, the present invention concerns a method of treatment, wherein the proteins are selected from the group consisting of the following protein families: lignin-forming anionic peroxidase, glucan endo-1,3-beta-glucosidase, endochitinase, pathogenesis-related protein, osmotin, proteinase inhibitors, and mixtures thereof.

In an embodiment, the present invention concerns a method of treatment, wherein the content, in the extract of tobacco leaves, of proteins whose molecular mass is greater than 100 kDa is less than 15% by weight based on the total protein weight of the dry extract.

In an embodiment, the present invention concerns a method of treatment, wherein the extract of tobacco leaves comprises at least one protein belonging to the family of glucan endo-1,3-beta-glucosidases selected from the group consisting of beta-1,3-endoglucanase acidic isoform PR-Q′ (PR36401 according to the UniProt database), beta-1,3-endoglucanase basic vacuolar isoform GLB (P27666 according to the UniProt database), and mixtures thereof.

In an embodiment, the present invention concerns a method of treatment, wherein the extract of tobacco leaves comprises at least one protein belonging to the family of endochitinases selected from the group consisting of acidic endochitinase P (P17513 according to the UniProt database), acidic endochitinase Q (P17514 according to the UniProt database), endochitinase B (P24091 according to the UniProt database), and mixtures thereof.

In an embodiment, the present invention concerns a method of treatment, wherein the extract of tobacco leaves comprises at least osmotin (P14170 according to the UniProt database).

In an embodiment, the present invention concerns a method of treatment, wherein the extract of tobacco leaves comprises at least one lignin-forming anionic peroxidase (P11965 according to the UniProt database).

In an embodiment, the present invention concerns a method of treatment, wherein the extract of tobacco leaves comprises at least one pathogenesis-related protein selected from the group consisting of-pathogenesis-related protein R (P13046 according to the UniProt database), pathogenesis-related protein PR-4A (PR29062 according to the UniProt database), pathogenesis-related protein PR-4B (PR29063 according to the UniProt database), and mixtures thereof.

In an embodiment, the present invention concerns a method of treatment, wherein the extract of tobacco leaves comprises at least one protein belonging to the family of proteinase inhibitors selected from the group consisting of proteinase inhibitor I-B (Q03199 according to the UniProt database), proteinase inhibitor I-A (Q03198 according to the UniProt database), and mixtures thereof.

In an embodiment, the present invention concerns a method of treatment, wherein the protein content in the dry extract of the extract of tobacco leaves is at least 10% by weight based on the total weight of the dry extract.

In an embodiment, the present invention concerns a method of treatment, wherein the proteins present in the extract of tobacco leaves are present in an amount ranging from 1 to 1000g/mL.

In an embodiment, the present invention concerns a method of treatment, wherein the extract of tobacco leaves or the composition is provided in a form suitable for administration by subcutaneous injection, in a form suitable for administration by means of an adhesive transdermal therapeutic system such as a patch, or in a form suitable for administration by spraying or by vaporization.

In an embodiment, the present invention concerns a method of treatment, wherein the extract of tobacco leaves or the composition is provided in a dosage form of 0.03 mL to 10 mL.

### Kit of Parts

As mentioned above a second aspect of the present disclosure concerns a kit comprising:
an aqueous solution of an enriched protein tobacco leaf extract; and
at least one product capable of controlling the symptoms of nicotine withdrawal.

As disclosed above for the method of treatment, preferably, the tobacco leaf extract contains at least 5% by weight, based on the total weight of the dry extract, of proteins of molecular mass greater than 10 kDa and essentially free of molecules of molecular mass less than 10 kDa.

More preferably, said proteins are selected from the group consisting of the following protein families: lignin-forming anionic peroxidase, glucan endo-1,3-beta-glucosidase, endochitinase, pathogenesis-related protein, osmotin and proteinase inhibitor and mixtures thereof.

The method for preparing said tobacco leaf extract is as disclosed above.

In an embodiment, the present invention concerns a kit, wherein the aqueous solution of an enriched protein tobacco leaf extract is suitable for a percutaneously or subcutaneously administration in an effective dose.

In an embodiment, the present invention concerns a kit, wherein said at least one product capable of controlling the symptoms of nicotine withdrawal is chosen from a nicotine agonist or partial agonist, a nicotine substitutes, or a combination thereof.

In an embodiment, the present invention concerns a kit, wherein said nicotine agonist or partial agonist is chosen from varenicline, cytisine, lobeline, or a combination thereof.

In an embodiment, the present invention concerns a kit, wherein the extract of tobacco leaves contains at least 5% by weight, based on the total weight of a dry extract thereof, of proteins of molecular mass greater than 10 kDa, and is essentially free of molecules of molecular mass less than 10 kDa and of RuBisCO proteins; and

wherein the content, in the extract of tobacco leaves, of proteins whose molecular mass is greater than 500 kDa, is less than 15% by weight, based on the total protein weight of the dry extract.

In an embodiment, the present invention concerns a kit, wherein the proteins are selected from the group consisting of the following protein families: lignin-forming anionic peroxidase, glucan endo-1,3-beta-glucosidase, endochitinase, pathogenesis-related protein, osmotin, proteinase inhibitors, and mixtures thereof.

In an embodiment, the present invention concerns a kit, wherein the content, in the extract of tobacco leaves, of proteins whose molecular mass is greater than 100 kDa is less than 15% by weight based on the total protein weight of the dry extract.

In an embodiment, the present invention concerns a kit, wherein the extract of tobacco leaves comprises at least one protein belonging to the family of glucan endo-1,3-beta-glucosidases selected from the group consisting of beta-1,3-endoglucanase acidic isoform PR-Q′ (PR36401 according to the UniProt database), beta-1,3-endoglucanase basic vacuolar isoform GLB (P27666 according to the UniProt database), and mixtures thereof.

In an embodiment, the present invention concerns a kit, wherein the extract of tobacco leaves comprises at least one protein belonging to the family of endochitinases selected from the group consisting of acidic endochitinase P (P17513 according to the UniProt database), acidic endochitinase Q (P17514 according to the UniProt database), endochitinase B (P24091 according to the UniProt database), and mixtures thereof.

In an embodiment, the present invention concerns a kit, wherein the extract of tobacco leaves comprises at least osmotin (P14170 according to the UniProt database).

In an embodiment, the present invention concerns a kit, wherein the extract of tobacco leaves comprises at least one lignin-forming anionic peroxidase (P11965 according to the UniProt database).

In an embodiment, the present invention concerns a kit, wherein the extract of tobacco leaves comprises at least one pathogenesis-related protein selected from the group consisting of-pathogenesis-related protein R (P13046 according to the UniProt database), pathogenesis-related protein PR-4A (PR29062 according to the UniProt database), pathogenesis-related protein PR-4B (PR29063 according to the UniProt database), and mixtures thereof.

In an embodiment, the present invention concerns a kit, wherein the extract of tobacco leaves comprises at least one protein belonging to the family of proteinase inhibitors selected from the group consisting of proteinase inhibitor I-B (Q03199 according to the UniProt database), proteinase inhibitor I-A (Q03198 according to the UniProt database), and mixtures thereof.

In an embodiment, the present invention concerns a kit, wherein the protein content in the dry extract of the extract of tobacco leaves is at least 10% by weight based on the total weight of the dry extract.

In an embodiment, the present invention concerns a kit, wherein the proteins present in the extract of tobacco leaves are present in an amount ranging from 1 to 1000 μg/m L.

In an embodiment, the present invention concerns a kit, wherein the extract of tobacco leaves or the composition is provided in a form suitable for administration by subcutaneous injection, in a form suitable for administration by means of an adhesive transdermal therapeutic system such as a patch, or in a form suitable for administration by spraying or by vaporization.

In an embodiment, the present invention concerns a kit, wherein the extract of tobacco leaves or the composition is provided in a dosage form of 0.03 mL to 10 mL.

According to an embodiment, the kit comprises one or more dose(s) of tobacco leaf extract, preferably in lyophilized form, as well as one or more dose(s) of saline solution or of WFI to prepare the pharmaceutical composition as described here-under just before administration.

According to an embodiment, the kit comprises one or more dose(s) of pharmaceutical composition ready to be administered to the patient.

The kit may optionally comprise one or more syringe(s) in order to administer the pharmaceutical composition by subcutaneous injection.

The kit may optionally comprise one or more patch(s) in order to administer the pharmaceutical composition transdermally.

### Tobacco Leaf Extract and Pharmaceutical Composition Thereof

The protein enriched tobacco leaf extract used in the here-above method of treatment and kit of parts is obtained as disclosed in WO2017174787.

Namely, said tobacco leaf extract may be obtained by a method of preparation comprising the following steps:
a. Curing of tobacco leaves,
b. Grinding of cured tobacco leaves to obtain ground cured tobacco leaves,
c. Extraction of the ground cured tobacco leaves under mechanical stirring with a solvent, for example an aqueous solvent, preferably an aqueous buffer solution with a pH between 6.0 and 8.5,
d. Separation of solid residues from the ground cured tobacco leaf extract solution by filtration or centrifugation to obtain a solid residue-free ground cured tobacco leaf extract solution,
e. Constant-volume filtration of the solution obtained in step d with a solvent, preferably an aqueous solvent, in an amount ranging from 2 to 12 times by volume, preferably from 3 to 10 times by volume, preferably from 4 to 8 times by volume, preferably 6 times by volume, based on the volume of the extract, and with a 10 kDa cut-off membrane,
f. Optionally lyophilization of the protein solution obtained in step e.

The dry extract may be obtained before lyophilization of the tobacco leaf extract according to the invention, then placed under a vacuum bell-jar, preferably in the presence of P₂O₅, until a constant mass of extract is obtained.

The curing of step a is carried out in open air, referred to as natural curing. In this case, curing is carried out in the presence of natural open air, outdoors or indoors. Curing can be carried out in an open or closed, covered or uncovered structure. Air-curing can for example be carried out in a natural curing barn. In this case, freshly harvested or pre-cured tobacco leaves are left to cure naturally under the effect of open air. The tobacco leaves can for example be hung in unheated ventilated barns. The tobacco leaves can for example be left to cure naturally until they turn brown. At this stage, there is practically no sugar left in the leaf. Advantageously, curing is carried out for a duration adapted to local climatic conditions, preferably for a minimum duration of one month. By way of example, curing can be carried out between September and December for harvests in central France. The tobacco leaves may be turned one or more times during curing, so as to allow uniform curing and to avoid the formation of condensation and the rotting or degradation of the leaves. The air-curing method can be used for example for Burley variety tobacco. According to a particular embodiment, the curing of step a is carried out in a natural curing barn.

For the purposes of the present invention, “air-curing” or “natural curing” means curing carried out in the presence of natural outdoor or indoor air. Air-curing can be carried out in an open or closed, covered or uncovered structure. Air-curing can for example be carried out in a natural curing barn. In this case, freshly harvested or pre-cured tobacco leaves are left to cure naturally under the effect of open air. The tobacco leaves can for example be hung in unheated ventilated barns. The tobacco leaves can for example be left to cure naturally until they turn brown. At this stage, there is practically no sugar left in the leaf. Advantageously, curing is carried out for a duration adapted to local climatic conditions, preferably for a minimum duration of one month. By way of example, curing can be carried out between September and December for harvests in central France. The tobacco leaves may be turned or aerated one or more times during curing, so as to allow uniform curing and to avoid the formation of condensation and the rotting or degradation of the leaves. The air-curing method can be used for example for Burley variety tobacco.

The tobacco leaf extract may be obtained from tobacco leaves cured in a natural curing barn.

The tobacco leaf extract may be obtained from tobacco leaves cured in a heated curing barn. In this case, curing can be carried out in a barn or a structure heated to a suitable temperature.

The tobacco leaf extract may be obtained from flue-cured tobacco leaves. In this case, curing can be carried out in a structure or a barn heated to a suitable temperature. Heat can be introduced into the structure or the barn through ducts connected to an external heat source. This controlled heating produces yellow-orange leaves. These leaves thus contain a high sugar content. Virginia tobacco can for example be cured according to this method.

The tobacco leaf extract may be obtained from sun-cured tobacco leaves. In this case, the tobacco leaves can be spread on racks and exposed to the sun for 12 to 30 days. Under direct light and heat from the sun, the leaves turn yellow or orange and retain a high sugar content. Oriental tobacco is generally cured using this method.

The tobacco leaf extract may be obtained from fire-cured tobacco leaves. In this case, small pieces of wood can be burned beneath the tobacco leaves, which cure while absorbing a “smoky” aroma.

According to one embodiment, the tobacco leaves are of the variety Nicotiana Tabacum or the variety Nicotiana Rustica. The tobacco leaves may come from brown or blond tobacco and may be selected from Virginia tobacco, Burley tobacco, Oriental tobacco, Latakia tobacco, Perique tobacco, Maryland tobacco, Kentucky tobacco, California tobacco, Tex Mex tobacco, and mixtures thereof.

Of course, the extract does not necessarily consist of a pure extract of tobacco leaves, and proteins extracted from cannabis can be added to treat both tobacco and cannabis dependence.

According to a particular embodiment, the tobacco leaf extract is obtained from a 1/1/1 blend of brown tobacco, Virginia tobacco and Burley tobacco.

According to a particular embodiment, the tobacco leaf extract is obtained from Burley tobacco.

According to one embodiment, the tobacco leaf extract is obtained from cured tobacco leaves.

According to a particular embodiment, the tobacco leaf extract is obtained from air-cured tobacco leaves.

According to a particular embodiment, the tobacco leaf extract is obtained from tobacco leaves air-cured for a duration adapted to local climatic conditions, preferably for a minimum duration of one month.

Steps c to e correspond to the extraction, filtration and diafiltration steps of the type described in U.S. Pat. No. 5,770,698 (column 6, line 47 to column 7, line 7) and patent application US 2009/0162403 (paragraphs [0032] to [0040]).

Preferably, the extraction in step c is carried out at a temperature between 4 and 20° C., preferably between 4 and 10° C.

Preferably, the extraction in step c is carried out for 12 to 36 h, preferably 22 to 26 h, preferably 24 h.

According to a particular embodiment, the solvent used in step c is an aqueous solvent. Preferably, the aqueous solvent used in step c is an aqueous buffer solution of ammonium bicarbonate, preferably at a concentration between 2 and 6 g/L, preferably 4 g/L.

Preferably, the tobacco leaf extract is obtainable by a solvent extraction process, for example an aqueous solvent, of ground cured tobacco leaves followed by separation of solid residues from the ground cured tobacco leaf extract solution then constant-volume diafiltration of the solid residue-free ground cured tobacco leaf extract solution with an aqueous solvent in an amount ranging from 2 to 12 times by volume, preferably 3 to 10 times by volume, preferably 4 to 8 times by volume, preferably 6 times by volume, based on the volume of the extract and with a 10 kDa cut-off membrane.

Of course, other types of solvent can be used to carry out the extraction process of the tobacco leaf extract of step c. The extraction of step c can be carried out with an organic or inorganic solvent, or with a mixture of organic and/or inorganic solvents.

To ensure good extraction efficiency and yield, and to obtain an extract of good quality with the desired purity properties and composition, the skilled person will easily know how to select the appropriate solvent(s) according to the following criteria:
the polarity of the solvent or solvent mixture (polar or non-polar);
the physical state of the solvent or solvent mixture (for example liquid, solid, supercritical or gaseous);
the chemical nature of the solvent or solvent mixture (for example organic or inorganic);
the charge of the solvent or solvent mixture (for example ionic or non-ionic);
the origin of the solvent or solvent mixture;
the miscibility of the solvent or solvent mixture; and/or
the solubility of the solvent or solvent mixture.

The skilled person will know which technique to use to select the solvent(s) corresponding to his criteria, based on the desired extraction yield and extract purity and composition.

Preferably, the extraction in step c is carried out by suspending the ground cured tobacco leaves in a buffer solution, preferably at a concentration of ground cured tobacco leaves in the buffer solution of 30 to 70 g/L, preferably 40 to 60 g/L, and more preferentially 50 g/L. The suspended solid residues are then removed by filtration, for example by Buchner filtration (step d), in order to obtain ground cured tobacco leaves free of solid residue. Preferably, the aqueous solvent used in step d is water for injection (WFI).

Preferably, the tobacco leaf extract may contain at least 5% by weight, based on the total weight of the dry extract, preferably at least 10% by weight, preferably at least 15% by weight, preferably about 20% by weight of proteins of molecular mass greater than 10 kDa and essentially free of molecules of molecular mass less than 10 kDa.

For the purposes of the present invention, “about” means plus or minus 1% due to measurement uncertainties.

Advantageously, step e removes more than 99% of molecules of molecular mass less than 10 kDa and notably free amino acids, proteins and peptides of molecular mass less than 10 kDa and protein residues of molecular mass less than 10 kDa resulting from the degradation of the proteins of step a. Advantageously, the content of molecules of molecular mass less than 10 kDa is less than 5% by weight based on the total weight of the extract, preferably less than 2.5% by weight, and better still less than 1% by weight.

The content of proteins of molecular mass greater than 10 kDa in the obtained extract usually measured in tobacco leaf extracts is about 1%. The tobacco leaf extract for the present invention therefore has a content of proteins of molecular mass greater than 10 kDa.

Said proteins in the protein enriched tobacco leaf extract are preferably selected from the group consisting of the following protein families: lignin-forming anionic peroxidase, glucan endo-1,3-beta-glucosidase, endochitinase, pathogenesis-related protein, osmotin and proteinase inhibitor and mixtures thereof.

The names indicated for the proteins present in the extract for the present invention correspond to the names given in the Swiss-Prot database, which is a biological database listing protein sequences.

According to one embodiment, the tobacco leaf extract for the present invention comprises at least one protein belonging to the family of glucan endo-1,3-beta-glucosidases, and preferably chosen from beta-1,3-endoglucanase acidic isoform PR-Q′ (PR36401 according to the UniProt database), beta-1,3-endoglucanase basic vacuolar isoform GLB (P27666 according to the UniProt database), and mixtures thereof.

According to one embodiment, the tobacco leaf extract for the present invention comprises at least one protein belonging to the family of endochitinases, and preferably chosen from acidic endochitinase P (P17513 according to the UniProt database), acidic endochitinase Q (P17514 according to the UniProt database), endochitinase B (P24091 according to the UniProt database), and mixtures thereof.

According to one embodiment, the tobacco leaf extract for the present invention comprises at least osmotin (P14170 according to the UniProt database).

According to one embodiment, the tobacco leaf extract for the invention comprises at least one lignin-forming anionic peroxidase (P11965 according to the UniProt database).

According to one embodiment, the tobacco leaf extract for the invention comprises at least one pathogenesis-related protein, and preferably chosen from pathogenesis-related protein R (P13046 according to the UniProt database), pathogenesis-related protein PR-4A (PR29062 according to the UniProt database), pathogenesis-related protein PR-4B (PR29063 according to the UniProt database), and mixtures thereof.

According to one embodiment, the tobacco leaf extract for the present invention comprises at least one protein belonging to the family of proteinase inhibitors, and preferably chosen from proteinase inhibitor I-B (Q03199 according to the UniProt database), proteinase inhibitor I-A (Q03198 according to the UniProt database), and mixtures thereof.

According to one embodiment, the tobacco leaf extract for the present invention also comprises polysaccharides of molecular mass greater than 10 kDa and preferably water-soluble.

Preferably, the tobacco leaf extract for the present invention comprises at least 5% by weight based on the total weight of the dry extract, preferably at least 10% by weight, preferably at least 15% by weight, preferably about 20% by weight of proteins selected from the group consisting of the following protein families: lignin-forming anionic peroxidase, glucan endo-1,3-beta-glucosidase, endochitinase, pathogenesis-related protein, osmotin and proteinase inhibitor and mixtures thereof.

According to one embodiment, the tobacco leaf extract for the present invention is essentially free of high molecular mass proteins.

For the purposes of the present invention, “high molecular mass protein” means a protein whose molecular mass is greater than 500 kDa, preferably whose molecular mass is greater than 400 kDa, more preferably whose molecular mass is greater than 300 kDa, preferentially whose molecular mass is greater than 200 kDa, even more preferentially whose molecular mass is greater than 150 kDa, and better still whose molecular mass is greater than 100 kDa.

According to one embodiment, the tobacco leaf extract for the present invention is essentially free of proteins whose molecular mass is greater than 50 kDa.

For the purposes of the present disclosure, “substantially free” means a content of molecules less than 15% by weight based on the total protein weight of the extract, preferably a content of molecules less than 10% by weight based on the total protein weight of the extract, more preferably less than 7.5% by weight based on the total protein weight of the extract, more preferably less than 5% by weight based on the total protein weight of the extract, preferentially less than 2.5% by weight based on the total protein weight of the extract, more preferentially less than 1% by weight based on the total protein weight of the extract, and better still less than 0.5% by weight based on the total protein weight of the extract.

Advantageously, the content of high molecular mass proteins is less than 15% by weight based on the total protein weight of the extract, preferably less than 10% by weight based on the total protein weight of the extract, more preferably less than 7.5% by weight based on the total protein weight of the extract, more preferably less than 5% by weight based on the total protein weight of the extract, preferentially less than 2.5% by weight based on the total protein weight of the extract, more preferentially less than 1% by weight based on the total protein weight of the extract, and better still less than 0.5% by weight based on the total protein weight of the extract.

According to one embodiment, the tobacco leaf extract for the present invention is essentially free of RuBisCO proteins. Advantageously, the curing of step a promotes the degradation of high molecular mass tobacco proteins, such as RuBisCO, for example by hydrolysis mechanisms.

Preferably, the protein solution obtained in step e is subjected to a step e′ of sterilizing filtration, prior to step f.

The tobacco leaf extract for the invention obtained at the end of the process described above may be stored as obtained at the end of step d′ of sterilizing filtration, or lyophilized as obtained at the end of step f.

The tobacco leaf extract for the present invention is advantageously an aqueous extract.

The tobacco leaf extract for the present invention is advantageously an aqueous extract that is brown in colour.

The present disclosure also concerns a pharmaceutical composition containing the enriched tobacco leaf extract for the present invention comprising as active principle the tobacco leaf extract as described above.

The pharmaceutical composition for the present invention may further comprise at least one pharmaceutically acceptable excipient, such as pharmaceutically acceptable solvents, and for example water.

Advantageously, the pharmaceutical composition for the present invention may comprise proteins present in the tobacco leaf extract in a content ranging from 1 to 1000 μg/mL, preferably from 10 to 500 μg/mL, preferably from 50 to 300 μg/mL, preferably from 60 to 200 μg/mL, preferably between 80 and 150 μg/mL.

The pharmaceutical composition for the present invention is preferably an aqueous composition.

According to an embodiment, the pharmaceutical composition for the present invention further comprises an adjuvant. Examples of adjuvants include swelling agents such as for example a sugar such as lactose, sucrose, trehalose, sorbitol, glucose, raffinose, mannitol, preferably lactose, sucrose, trehalose, glucose, or mannitol, an amino acid such as arginine, glycine or histidine, preferably glycine, or polymers of dextran or polyethylene glycol type, or mixtures thereof. According to this embodiment, the pharmaceutical composition comprises 50 to 99% by weight of adjuvants, preferably 80 to 97% by weight, based on the total weight of the pharmaceutical composition.

According to a particular embodiment, the pharmaceutical composition for the present invention may further comprise proteins extracted from cannabis.

Preferably, the pharmaceutical composition may be prepared by a process comprising the following steps:
a1. Preparation of a tobacco leaf extract as defined above,
b1. Adjustment of the protein concentration so as to obtain a protein concentration ranging from 100 to 200 μg/mL,
c1. Addition of the pharmaceutically acceptable excipient(s), and
d1. Optionally, addition of adjuvant(s) to this extract, preferably addition of mannitol.

The tobacco leaf extract used in step b1 can be either that obtained directly after the step of diafiltration on a 10 kDa cut-off membrane, or that obtained after the step of sterilizing diafiltration, or the tobacco leaf extract which is lyophilized then reconstituted for example in saline solution or water.

Advantageously, the concentration in step b1 can be adjusted:
either by dilution with water in order to lower the protein concentration, or
by diafiltration in order to increase the protein concentration.

A pharmaceutically acceptable excipient particularly preferred in step c1 is water.

The adjuvants that can be added in step d1 may be notably swelling agents such as for example a sugar such as lactose, sucrose, trehalose, sorbitol, glucose, raffinose, mannitol, preferably lactose, sucrose, trehalose, glucose, or mannitol, an amino acid such as arginine, glycine or histidine, preferably glycine, or polymers of the dextran or polyethylene glycol type; or mixtures thereof.

According to a particular embodiment, step d1 comprises at least the addition of mannitol.

According to one embodiment, the pharmaceutical composition for the present invention is provided in a form suitable for subcutaneous administration.

According to one embodiment, the pharmaceutical composition for the present invention comprises an adhesive transdermal therapeutic system containing the tobacco leaf extract as defined above. According to this embodiment, the pharmaceutical composition for the present invention is provided in a form suitable for administration by means of an adhesive transdermal therapeutic system. Advantageously, the pharmaceutical composition for the present invention is provided in a patch form.

The patch can be in the form of a reservoir-type patch with one or more compartments or a matrix-type patch. The practical execution of the patches will be determined by the skilled person based on his general knowledge of the subject to obtain a controlled and prolonged systemic administration of the tobacco leaf extract, over the entire period of application of the patch, for example for a duration of about 2 h to 24 h.

The reservoir-type patch will comprise one or more separate reservoir(s) containing the active principle(s) (including the tobacco leaf extract) in solution or suspended in the polymer matrix coming into contact with the skin via a semi-permeable polymeric membrane allowing the rate of release of the active principle(s) to be adjusted.

The matrix-type patch will comprise a polymeric mass within which the active principle(s) will be dissolved or dispersed in the appropriate proportions. These active principles are released by diffusion through the polymer chains of said matrix.

According to a particular embodiment of this type of patch, the adhesive covers the entire release surface of the matrix and is an integral part of the latter. This is therefore an active adhesive-type patch well known to the skilled person, which is of simplified manufacture and which allows the creation of thin, suitably flexible patches allowing comfortable application on the patient's skin.

To ensure a good infusion of these active principles subcutaneously, or in the bloodstream, at the appropriate dose, at the appropriate diffusion rate, and for the appropriate duration, the skilled person can easily set the following parameters:
the ratio of the surface areas and the volumes of each compartment of the patch;
the optional addition of one or more hydrophilic additive(s);
the optional addition of one or more diffusion activator(s) or inhibitor(s);
the optional addition of one or more solubilizer(s);
the optional addition of one or more stabilizer(s);
the optional addition of one or more absorption promoter(s), and
more generally, all types of additives well known to the skilled person allowing good control of the flow and stability of the tobacco leaf extract.

The skilled person will know which technique to use to set the parameters listed above based on the desired solubility and stability.

The various manufacturing parameters of the patch will be easily adapted by the skilled person to arrive at the desired dosage.

In a manner known per se, such a patch comprises a removable protective film which is intended to preserve the adhesive side to be applied to the skin after the patch has been manufactured and throughout its storage period. In a manner known per se, the skilled person will for example use polyester films, one side of which can be treated with anti-adherent silicones.

According to one embodiment, the composition for the present invention is administered by subcutaneous injection. According to this embodiment, the pharmaceutical composition is provided in a form suitable for subcutaneous administration.

According to one embodiment, the pharmaceutical composition is provided in a dosage form of 0.03 mL to 10 mL, preferably 0.1 mL to 5 mL, preferably 0.5 to 2 mL. The pharmaceutical composition is thus preferably administered in an amount of 0.03 mL to 10 mL, preferably 0.1 to 5 mL, preferably 0.5 mL to 2 mL by subcutaneous injection.

The pharmaceutical composition may be provided in a form intended to be administered by subcutaneous injection only once.

The pharmaceutical composition may be provided in a form intended to be administered by subcutaneous injection several times, preferably two or three times.

In the latter case, the pharmaceutical composition is preferably administered by subcutaneous injection on days 0 and 10 or on days 0, 10 and 30.

The dose injected the second time, and optionally the third time, may be identical to the dose injected the first time.

### FIGURES

FIG. 1 shows in a graph the reduction in smoking satisfaction with NFL-101 and the placebo, that was obtained in the PRECESTO study (see examples).

FIG. 2 shows in a graph the difference between the reduction in smoking satisfaction with NFL-101 and the placebo, including the exploratory p-values, that was obtained in the PRECESTO study (see examples).

FIG. 3 shows in a graph the results of a comparative test on the reduction of smoking satisfaction between Champix® (i.e., Varenicline, an alternative solution found on the market) and NFL-101.

### EXAMPLES

NFL-101 is an aqueous extract of tobacco leaves prepared according to Good Manufacturing Practice guidelines. This subcutaneous injectable solution is available as lyophilized powder (140 μg of proteins per vial) and accompanied by a sterile dilution solution. The vial will be reconstituted by the study site on the day of injection to ensure stability of the product.

The process of manufacturing NFL-101 complies with the teaching of WO2017174787. Therefore, for the sake of completeness of the present invention's disclosure, example 1 of WO2017174787 is disclosed here-under for the reproduction of the present invention.

### Example 1: Preparation of a Tobacco Leaf Extract According to the Invention (WO2017174787)

Burley tobacco leaves are used to prepare the extract.

100 g of Burley tobacco leaves cured naturally for about three months (in central France, between September and December) are ground. The ground leaves are suspended for 24 h at a temperature of 4 to 10° C. in 1 892 g of WFI to which 8 g of ammonium bicarbonate has been added. The suspended solid residues are then removed by Büchner filtration.

A 0.2 μm clarifying filtration of the extract is then carried out. The extract obtained is brown in colour. The extract is then weighed to determine the volume of WFI to be used in the constant-volume diafiltration step; in this case the mass of the liquid extract is 1 680 g.

An extraction of the proteins of this solid residue-free ground cured tobacco leaf extract is then performed: 10 080 g of WFI is then added to the solid residue-free ground cured tobacco leaf extract. The 11 760 g of solution thus obtained is diafiltered at constant volume against 6 times the volume with a 10 kDa cut-off (MerckMillipore) until the mass of the retentate is reduced to 1 680 g.

The retentate obtained after diafiltration is a protein mixture in which proteins of molecular mass less than 10 kDa are detected with a concentration 99% lower than their initial concentration. The colour of the retentate obtained is between B2 and B3 according to the measurement scale defined in EUROPEAN PHARMACOPOEIA 5.0, 2.2.2, Degree of coloration of liquids.

This diafiltrate can be lyophilized, after addition of mannitol for example, using an SMH 150 lyophilizer, to obtain a lyophilized tobacco leaf extract.

NFL-101 is an aqueous extract of tobacco leaves.

NFL- 101 (extract of example 1) is presented in the form of 1 vial of 140 μg of sterile lyophilized powder and 1 vial of 5 ml of NaCl 0.9%.

Vial must be reconstituted in 1.4 or 2.8 mL of NaCI 0.9%, obtaining a solution at the concentration of 50 μg/mL or 100 μg/mL. NFL-101 reconstituted solution has a light yellow-brown color.

### Example 2: Clinical Development

a. Phase I Clinical Trial (CESTO Trial)

A Phase I trial was conducted in 24 smoking subjects (average Fagerstom: 6.25). The primary objective was to evaluate the safety of NFL-101. The secondary objectives were to assess immunogenicity and efficacy of NFL-101 in these smoking subjects. The study was a prospective monocentre, open-label and non-randomized phase I trial conducted at the Institut Regional du Cancer (ICM) in Montpellier (France).

NFL- 101 was subcutaneously administered twice, with an interval of 4 weeks, to smoking subjects willing to quit. Each subject received 2 injections (1 in each arm) at D1 and D29. The study aimed to investigate 2 levels of proteins (100 μg or 200 μg of proteins), to determine whether both dose levels were safe and which dose was the most effective. Subjects were included according to a 6+6 dose escalation design. The first 12 subjects (6 doses 100 μg of proteins+6 doses 200 μg of proteins) received the treatment a few days after the recruitment visit. The last 12 subjects had more time to prepare their quit attempt before the treatment administration and as a result were noted to have better psychological preparation for tobacco cessation.

The dose-limiting toxicity (DLT) in this study was defined as any Grade 3-4 toxicity according to NCI-CTCAE V4.0 classification. No DLTs were observed among any subjects. Only 1 Grade 2 toxicity (anxiety) that was deemed to be related or probably related to NFL-101 treatment was reported at Dose Level 2 (higher dose). Other adverse events noted were the following Grade 1 toxicities:
n=20: pain at injection site, deltoid pain, deltoid oedema, bilateral pain (10 at Dose 1 [lower dose] and 10 at Dose 2 [higher dose]);
n=7: dry mouth (3 at Dose 1 and 4 at Dose 2);
n=6: tiredness (3 at Dose 1 and 3 at Dose 2);
n=5: nausea (1 at Dose 1 and 4 at Dose 2);
n=4: dry eyes (3 at Dose 1 and 1 at Dose 2);
n=3: insomnia (2 at Dose 1 and 1 at Dose 2);
n=3: erythema deltoid (1 at Dose 1 and 2 at Dose 2);
n=2: shivers (1 at Dose 1 and 1 at Dose 2);
n=2: irritability (1 at Dose 1 and 1 at Dose 2);
n=2: cephalalgia (1 at Dose 1 and 1 at Dose 2);
n=2: dysgeusia (2 at Dose 1 and 0 at Dose 2);
n=2: restlessness (1 at Dose 1 and 1 at Dose 2);
n=1: bradycardia, photophobia, diarrhea, tongue pruritus, gastroesophageal reflux, weight gain, calf cramps, pain (deltoid extremities), anxiety, depression, clear rhinitis, nasal xerosis, productive cough, pruritus (arm and legs), pruritus (nasal).

During the study, 67% of the 24 smokers reduced their cigarette consumption by at least 50% at 4 weeks, and 63% at 12 weeks. 33% of the 24 smokers did not smoke for at least the last 7 days at 12 weeks. 21% of the 24 smokers maintained continuous abstinence at 12 weeks. 80% of the last 12 subjects who had better psychological preparation for the tobacco cessation trial reduced their consumption by at least 50%, and 5 of them were continuously abstinent 8 weeks after the end of the treatment (at 12 weeks).

Immunology assessment showed a significant increase of anti-NFL101 IgG (already present at D1) and correlations between higher anti-NFL-101 IgG (after treatment administration) and smoking reduction, 7 days' point prevalence abstinence and continuous abstinence.

All subjects spontaneously reported that NFL-101 decreased their desire to smoke and cigarette appetence but reported experiencing classic withdrawal symptoms during their quit attempts.

### Conclusion of the CESTO Trial

People who try to stop smoking typically experience urges to smoke, often described as cravings, frequently in response to environmental cues associated with smoking (such as drinking alcohol). These urges decrease in intensity and frequency with time. The key to stopping smoking is to resist these urges. Effective medication for smoking cessation reduces the intensity of the urges and this is the likely mechanism of action.

There are three licensed medications for smoking cessation: bupropion, varenicline, and nicotine replacement therapy (NRT). Bupropion has rather more contraindications to its use and results in more severe adverse effects than the other two medications. Thus, when people who smoke use medication to assist them, they typically use either varenicline or NRT.

Conventionally, people stopping smoking commence NRT on quit day.

The most effective medication for supporting smoking cessation is varenicline. Varenicline is a nicotinic partial agonist and it is therefore surprising to find that it is more effective than a full agonist, i.e., nicotine.

An investigation considered the possible mechanisms of action and compared varenicline, bupropion, and a placebo. It is found that varenicline reduced urges to smoke to a lower level than did bupropion.

Furthermore, varenicline leads to lower satisfaction from a lapse (smoking episode) after quit day than does bupropion. However, many other mood-related symptoms of withdrawal are of similar intensity. This suggests that a key mechanism of action of the smoking cessation pharmacotherapies, which relates directly to efficacy, is controlling urges and reducing satisfaction from smoking.

Unlike NRT, varenicline is used for one to two weeks prior to quit day, which might explain its superior efficacy. Clinical trials have studied if nicotine preloading, meaning the use of NRT prior to quitting smoking while continuing either to try to smoke the same amount or to smoke freely, increases abstinence.

A meta-analysis of nicotine preloading for smoking cessation including eight relevant studies, with 2,813 participants, concluded on a weak non-significant effect of nicotine preloading on abstinence. Another study on 1792 smokers concluded that use of nicotine patch preloading prior to attempting to stop smoking can increase the proportion of people who stop successfully, but its benefit is undermined because it reduces the use of varenicline after preloading.

The question to be answered is whether NFL-101 could significantly reduce satisfaction from smoking in order to be used prior to quitting.

b. NFL-101 Preconditioning Combined With NRT Treatment

Since NRT is one on the two medications preferably used for smoking cessation and is not currently used as a preconditioning prior to quit day nor has clearly demonstrated its efficacy as a preconditioning, it is of interest to examine if using NFL-101 as a preconditioning prior to quit day and to commence NRT could improve smoking cessation.

We may hypothesise the potential mechanism of NFL-101 preconditioning followed by NRT treatment by understanding the development of tobacco addiction.

Tobacco smoking creates a positive reward, such as feelings of pleasure, which leads to the reinforcement of subsequent smoking. After a period of regular smoking, neuroadaptation to regular doses of nicotine means that people who smoke, experience negative moods when nicotine concentrations in the brain drop; these feelings are relieved by smoking and, thus, smoking is negatively reinforced.

Independently of any reward that people may experience, mechanisms in the nucleus accumbens set up associative learning that results in the drive to smoke. These mechanisms create an acquired drive to smoke and tobacco dependence, manifested by the regular consumption of cigarettes, the occurrence of tobacco withdrawal symptoms when unable to smoke and difficulty in stopping smoking if a person were to choose to do so. The net effect of the changes in the drive to smoke mean that a person will not smoke when he or she normally would, usually when cued by the environment to do so. Not smoking when cued to smoke will begin to extinguish the learnt association between the action of smoking a cigarette and reinforcement in the brain.

Aside from this addiction-based mechanism, three other mediational hypotheses could be advanced for a mechanism of action of NFL-101 in a preconditioning.

The first hypothesis is that a preconditioning could increase post quit day medication adherence.

The second hypothesis is that the reduced level of smoking that occurs when people receive the NFL-101 may increase a person's confidence that they can abstain from smoking after quit day; the confidence that one can achieve abstinence is associated with achieving abstinence.

The third hypothesis is that aversive reaction to cigarettes after receiving NFL-101 may increase the rate of cessation. Aversive smoking is a process in which people smoke excessively to the point of nausea and vomiting. Although it is rarely used as a treatment to enhance cessation, it is effective at increasing the rate of cessation.

c. Precesto Study

### c.1. Historically

It was thus proposed to conduct a Phase Ila trial (so-called the PRECESTO study) to assess the efficacy of NFL-101 compared to a placebo to reduce the reward from smoking. A questionnaire “mCEQ” is used as the main assessment criteria in a crossover design on 34 patients smoking ad libitum. Subjects were to be advised that they were to receive both active (200 μg NFL-101) and placebo study medication but would remain blinded to whether they would receive NFL-101 or placebo at the first and second period. A duration of the periods of 4 weeks was made possible to clearly observe the evolution of the effect over time. It would be considered that the washout period of 30-day minimum would be long enough to avoid any remanent effect when starting the second period.

The primary endpoint would have been the success in achieving a 1 point reduction in the mCEQ “Smoking Satisfaction” subscale measured on D4 (day 4), from the first administration of NFL-101. The mCEQ would have comprised all twelve questions mentioned above with a 7 points scale.

A secondary criteria to be appreciated would have been achieving a 1 point reduction in:
mCEQ “Smoking Satisfaction” subscale measured at D7, D14, D21 and D28, and/or
mCEQ “Psychological Reward”; “Aversion”; “Enjoyment of Respiratory Tract Sensations”; and “Craving Reduction” subscales, measured at D4, D7, D14, D21 and D28.

To this, other questions would have been presented to the subjects to assess the effectiveness of the treatment:
“Have you found your urges to smoke stronger or weaker than usual in the last 24 hours?” (with response options of “much stronger,” “slightly stronger,” “same as before,” “slightly weaker,” and “much weaker”) at D4, D7, D14, D21 and D28?
“Have you found cigarettes more or less enjoyable than usual in the last 24 hours?” (with response options of “much more enjoyable,” “slightly more enjoyable,” “same as before,” “slightly less enjoyable,” and “much less enjoyable”) at D4, D7, D14, D21 and D28?
Motivation to stop scale? (from 01 to 10, 10 representing an urge to stop)?
Number of cigarettes smoked in the last 24hours?

Moreover, over criteria due to nicotine withdrawal could have been appreciated (depression, insomnia, irritability/frustration/anger, anxiety, difficulty concentrating, restlessness, increased appetite/weight gain, constipation, bradycardia . . . ). These were the initial milestones planned in the completion of the CESTO study.

### c.2. Conditions and Implementation of the Conducted Studies

In concrete terms, PRECESTO was thus the second study after CESTO which highlighted the effect of NFL-101 in terms of reducing smoking satisfaction. The clinically relevant effect was more prolonged than expected.

These results confirm the therapeutic interest of NFL-101 administered on its own or in combination with nicotine replacement therapies, which reduce withdrawal symptoms. They thus corroborate the previous results of the CESTO study.

### Recap: What Is Precesto?

PRECESTO is a monocentric, placebo-controlled, randomized and double-blind Phase 2a exploratory study, with a crossover covering two periods of 28 days each, including 34 smokers who do not want to quit and have high smoking satisfaction. Each participant is his or her own control and receives, on a random and alternating basis, either NFL-101 or the placebo at the start of each of the two periods. At the end of the study, all the participants received one dose of NFL-101 and one dose of the placebo. For each participant, the order in which NFL-101 and the placebo were administered was kept secret. For each period, the participants received the treatments on day 1 (D1) then answered, with complete independence, the modified Cigarette Evaluation Questionnaire (mCEQ) on days 4 (D4), 7(D7 ), 14 (D14), 21(D21 ) and 28(D28 ).

The aim of PRECESTO is to “assess the efficacy of NFL-101 to reduce the positive reinforcement of cigarettes as measured by the subscale assessing smoking satisfaction (questions 1, 2 and 12) from the mCEQ versus a placebo”. The primary criterion resulting from this objective was chosen as “the Smoking Satisfaction subscale (items 1, 2 and 12) from the mCEQ measured on D4”.

mCEQ, A Leading International Questionnaire for Measuring the Effects of Smoking

The mCEQ is an international questionnaire that is completed independently. The study's participants, smokers who have high smoking satisfaction and do not wish to quit, answer 12 questions marking the number that best represents how smoking makes them feel (1-not at all, 2-very little, 3-a little, 4-moderately, 5-a lot, 6- quite a lot, 7- extremely). The questions are as follows:
1. Was smoking satisfying?
2. Did cigarettes taste good?
3. Did you enjoy the sensations in your throat and chest?
4. Did smoking calm you down?
5. Did smoking make you feel more awake?
6. Did smoking make you feel less irritable?
7. Did smoking help you concentrate?
8. Did smoking reduce your hunger for food?
9. Did smoking make you dizzy?
10. Did smoking make you nauseous?
11. Did smoking immediately relieve your craving for a cigarette?
12. Did you enjoy smoking?

The questionnaire uses three multi-item subscales and two single items: “Smoking Satisfaction” (items 1, 2, and 12); “Psychological Reward” (items 4 through 8); “Aversion” (items 9 and 10); “Enjoyment of Respiratory Tract Sensations” (item 3); and “Craving Reduction” (item 11). Scores for each subscale are calculated as the mean of the individual item responses or the single item. Each score may therefore vary from 1 to 7. Higher scores indicate greater intensity on that scale.

The participants in the PRECESTO study completed this questionnaire themselves remotely using their computer. They were asked to refer to the cigarette that they smoked the previous day after dinner in order to standardize, insofar as possible, the change in perceptions over time for each subject.

### Rationale for Conducting the Precesto Study

The PRECESTO study makes it possible to better understand the activity of NFL-101 and specifically this product's efficacy in terms of reducing smoking satisfaction.

In the fight against smoking, reducing smoking satisfaction is essential: to successfully stop smoking, it is vital to tackle both smoking satisfaction and nicotine withdrawal. In terms of withdrawal, there are nicotine replacement therapies, but to tackle satisfaction, there are no effective medicinal products that do not have side effects.

### c.3. Results

### PRECESTO Phase 2a Exploratory Clinical Study

It was previously verified that the statistical analysis could be carried out on all subjects and for the 2 sequences jointly (carryover effect: p>0,10). The data are processed by calculating the least squares mean taking into account the study's crossover design and in accordance with the PRECESTO protocol.

Smoking satisfaction is the average for the answers to questions 1, 2 and 12 from the questionnaire. On inclusion, the average score for smoking satisfaction was 6.12 (standard deviation 0.77).

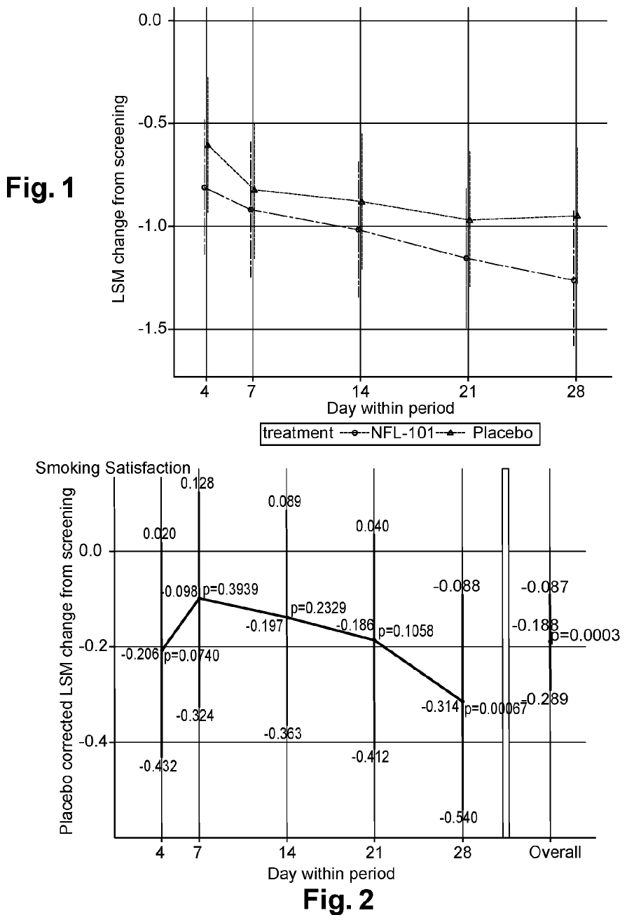

The precesto results are as follows:
Concerning the reduction in smoking satisfaction generated by NFL-101: According to the observations from the CESTO Phase 1 clinical trial, a rapid effect reaching a maximum level at D4 then dissipating over 10 days was expected. The effect observed with PRECESTO is different (FIG. 1): there is an effect on D4 (average reduction −0.810; confidence interval (−1.139; −0.481)) and this effect then grows stronger on a relatively linear basis through to the final measurement on D28 (average reduction −1.261; confidence interval (−1.590; −0.932)). The average level of smoking satisfaction which, on inclusion, was between quite a lot and extremely, was reduced to between moderately and a lot on D28, which represents a clinically relevant reduction.
Concerning the effect of NFL-101 versus placebo and the reduction in smoking satisfaction: The placebo effect is significant, but still lower than the effect of NFL-101 throughout the observation (FIG. 1 below). It increases less quickly from D7, then decreases from D21 through to D28 (average reduction −0.947; confidence interval (−1.277; −0.618)). Regarding the difference in effects between NFL- 101 and the placebo (FIG. 2), this did not reach significance on D4 (>0.05) and the exploratory p-values are 0.0003 (average difference −0.188; confidence interval (−0.289; −0.087)) over the entire period monitored from D4 to D28 and 0.007 (average difference −0.314; confidence interval (−0.540; −0.088) on D28.
Concerning the specific effect on the other mCEQ subscales and items in terms of secondary criteria: Exploratory p-values have not been calculated for these criteria. The limitations of the study, which are presented in detail below, may provide an explanation, particularly for the criterion relating to craving.

FIG. 1 shows in a graph the reduction in smoking satisfaction with NGL-101 and the placebo. The smoking satisfaction (ordinate) is the Least Square Mean (LSM) change from screening (pool of periods 1 and 2) with 95% CI from MMRM. It can clearly be seen on the graph that although a placebo effect on the smoking satisfaction is seen, NFL-101 (active ingredient) does prove a greater effect on the smoking satisfaction reduction in comparison to the Placebo.

FIG. 2 shows in a graph the difference between the reduction in smoking satisfaction with NFL-101 and the placebo, including the exploratory p-values. The smoking satisfaction (ordinate) is the placebo corrected Least Square Means (LSM) change from screening (Pool of periods 1 and 2) with 95 CI and exploratory p-values from MMRM including the period, the sequence, all the visits, and, as covariates, the sex and the value at screening. The graph proves that on a period ranging up to 28 days, the smoking satisfaction decreases meaningfully even in view of the placebo effect.

Comparison with varenicline (Chantix®/Champix®):

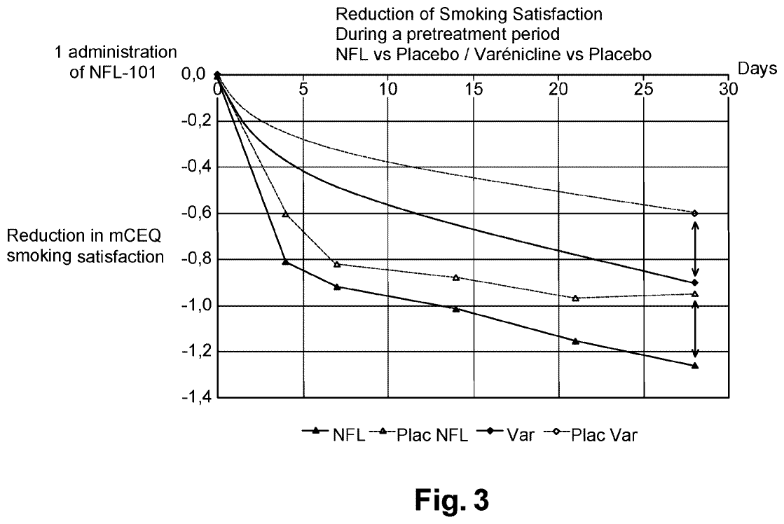

To better perceive the therapeutic potential of NFL-101 it is interesting to compare the reduction in smoking satisfaction obtained in PRECESTO with those obtained by varenicline in pretreatment conditions, that is to say before an attempt to stop (Bohadana A, Freier-Dror Y, Peles V, Babai P, Izbicki G. Extending varenicline preloading to 6 weeks facilitates smoking cessation: A single-site, randomised controlled trial. EClinicalMedicine. 2020 Feb. 3;19:100228. doi: 10.1016/j.eclinm.2019.11.021. PMID: 32055787; PMCID: PMC7005428). Over 4 weeks, a single administration of NFL- 101 reduced smoking satisfaction more than 53 administrations of varenicline as it can be reported in FIG. 3. Thus, on FIG. 3, the reduction of smoking satisfaction during a pretreatment period with NFL vs. Placebo is directly compared to that of varenicline (Champix ®) vs Placebo, based on the clinical trial PRECESTO for one and the here above cited publication (Bohadana A et al.). The Champix® vs Placebo results presents a difference of 0,3(p=0,04 ), on the basis of 53 administrations of Champix®. The NFL- 101® vs Placebo results presents a difference of 0,36(p=0,007 ) in the conditions set above. The reduction of smoking satisfaction from baseline is greater with NFL-101 (−1.26) than with Champix® (−0,9).

### Clarifications Concerning the P-Values

For the p-values of 0.0003 and 0.007, this means that if there were no real effects or differences in the data, there would be just a 0.03% and 0.7% chance of obtaining data that were as different as those observed.

The calculation of the exploratory p-values carried out with a post hoc analysis was limited to the study's primary objective. This approach makes it possible to reduce the risks of obtaining low p-values resulting from chance, which may be the case when p-values are calculated on a number of criteria or even by cross-referencing the criteria in relation to one another. These calculations make it possible to adapt to the observation of a more prolonged effect than expected, while respecting the spirit of the protocol and its primary objective.

### Limitations of the Precesto Study

With the PRECESTO study, the mCEQ was completed by smokers who did not want to quit and who smoked ad libitum (“as often as desired”). It is recognized that reducing the consumption of cigarettes or stopping smoking generates significant effects on the perceptions and the behavioural responses associated with cigarette consumption. The crossover design did not make it possible to include subjects who were going to reduce their consumption or make attempts to stop as this would have resulted in a too significant carry-over effect (persistence or continuation of an effect) from period 1 to period 2. In addition, recruiting subjects who wanted to stop smoking would have involved the risk of information loss if they stopped, because subjects who stop smoking can no longer assess their level of smoking satisfaction and therefore complete the questionnaire. By recruiting smokers who do not want to stop smoking, it was more difficult to show an effect for NFL-101 on certain criteria such as craving as this was regularly reduced by the cigarettes smoked without any constraints.

### c.4.Conclusion: A First Placebo-Controlled Clinical Trial With Promising Results

This exploratory study:
shows that a single administration of NFL-101 reduces smoking satisfaction in a clinically relevant way and with a more prolonged effect than expected, which confirms the therapeutic interest of NFL-101 administered on its own or in combination with nicotine replacement therapies, which reduce withdrawal symptoms;
represents a second clinical trial after CESTO revealing an effect on reducing smoking satisfaction, and on a placebo-controlled basis this time, the level of proof of the effect of NFL-101 is strengthened; and
corroborates the clinical results already mentioned previously (CESTO trial).

1-57. (canceled)

58. An aqueous solution comprising an enriched protein tobacco leaf extract, essentially devoid of nicotine, said aqueous solution for a preconditioning method of a subject to cess smoking, said aqueous solution configured to be administered to the subject percutaneously or subcutaneously in an effective dose of said aqueous solution of an enriched protein tobacco leaf extract, during which the subject continues to smoke ad libitum.

59. The aqueous solution of an enriched protein tobacco leaf extract, essentially devoid of nicotine, according to claim 58, wherein the said aqueous solution configured to be administered to the subject percutaneously or subcutaneously in a second effective dose of said aqueous solution of an enriched protein tobacco leaf extract, essentially devoid of nicotine, during which the subject continues to smoke ad libitum.

60. The aqueous solution of an enriched protein tobacco leaf extract, essentially devoid of nicotine, according to claim 59, wherein the second effective dose of ana aqueous solution of an enriched protein tobacco leaf extract, essentially devoid of nicotine, is configured to be administered within 2 to 15 days, from the first administration.

61. The aqueous solution of an enriched protein tobacco leaf extract, essentially devoid of nicotine, according to claim 58, wherein the extract of tobacco leaves contains at least 5% by weight, based on the total weight of a dry extract thereof, of proteins of molecular mass greater than 10 kDa, and is essentially free of molecules of molecular mass less than 10 kDa and of RuBisCO proteins; and
wherein the content, in the extract of tobacco leaves, of proteins whose molecular mass is greater than 500 kDa, is less than 15% by weight, based on the total protein weight of the dry extract.

62. The aqueous solution of an enriched protein tobacco leaf extract, essentially devoid of nicotine, according to claim 58, wherein the extract of tobacco leaves or the composition is provided in any one of: a form suitable for administration by subcutaneous injection, in a form suitable for administration by means of an adhesive transdermal therapeutic system such as a patch, or in a form suitable for administration by spraying or by vaporization.

63. The aqueous solution of an enriched protein tobacco leaf extract, essentially devoid of nicotine, according to claim 58, wherein the extract of tobacco leaves or the composition is provided in a dosage form of 0.03 mL to 10 mL.

64. A method of administering an aqueous solution of an enriched protein tobacco leaf extract, essentially devoid of nicotine, for the treatment of smoking cessation of a subject, comprising the following successive steps:
i) a preconditioning step during which the subject is administered percutaneously or subcutaneously at least one effective dose of an aqueous solution of an enriched protein tobacco leaf extract, essentially devoid of nicotine, during which the subject continues to smoke ad libitum, and
ii) a second step following the preconditioning step i), within 2 to 180 days from said administration to the subject carried out during the preconditioning step i), during which the subject completely quits smoking with one or several optional complementary administrations of at least one product capable of controlling the symptoms of nicotine withdrawal.

65. The method of administering an aqueous solution of an enriched protein tobacco leaf extract, essentially devoid of nicotine, according to claim 64, wherein the preconditioning step comprises two or three percutaneous administrations of effective doses of two or three aqueous solutions of an enriched protein tobacco leaf extract, essentially devoid of nicotine, during which the subject continues to smoke ad libitum.

66. The method of administering an aqueous solution of an enriched protein tobacco leaf extract, essentially devoid of nicotine, according to claim 65, wherein the two or three percutaneous administrations of effective doses of two or three aqueous solutions of an enriched protein tobacco leaf extract, essentially devoid of nicotine, during which the subject continues to smoke ad libitum, are separated one from each other by at least 10 days.

67. The method of administering an aqueous solution of an enriched protein tobacco leaf extract, essentially devoid of nicotine, in the treatment according to claim 64, wherein the proteins present in the extract of tobacco leaves are present in an amount ranging from 1 to 1 000 μg/m L.

68. The method of administering an aqueous solution of an enriched protein tobacco leaf extract, essentially devoid of nicotine, according to claim 64, wherein the extract of tobacco leaves or the composition is provided in a form suitable for administration by subcutaneous injection, in a form suitable for administration by means of an adhesive transdermal therapeutic system such as a patch, or in a form suitable for administration by spraying or by vaporization.

69. The method of administering an aqueous solution of an enriched protein tobacco leaf extract, essentially devoid of nicotine, according to claim 64, wherein the extract of tobacco leaves or the composition is provided in a dosage form of 0.03 mL to 10 mL.

70. A kit comprising:
an aqueous solution of an enriched protein tobacco leaf extract; and separately
at least one product capable of controlling the symptoms of nicotine withdrawal.

71. The kit according to claim 70, wherein the aqueous solution of an enriched protein tobacco leaf extract is suitable for a percutaneously or subcutaneously administration in an effective dose.

72. The kit according to claim 70, wherein said at least one product capable of controlling the symptoms of nicotine withdrawal is chosen from: a nicotine agonist or partial agonist, a nicotine substitutes, or a combination thereof.

73. The kit according to claim 72, wherein said nicotine agonist or partial agonist is chosen from:
varenicline, cytisine, lobeline, or a combination thereof.

74. The kit according to claim 70, wherein the extract of tobacco leaves contains at least 5% by weight, based on the total weight of a dry extract thereof, of proteins of molecular mass greater than 10 kDa, and is essentially free of molecules of molecular mass less than 10 kDa and of RuBisCO proteins; and
wherein the content, in the extract of tobacco leaves, of proteins whose molecular mass is greater than 500 kDa, is less than 15% by weight, based on the total protein weight of the dry extract.

75. The kit according to claim 70, wherein the proteins present in the extract of tobacco leaves are present in an amount ranging from 1 to 1 000 μg/m L.

76. The kit according to claim 70, wherein the extract of tobacco leaves or the composition is provided in any one of: a form suitable for administration by subcutaneous injection, in a form suitable for administration by means of an adhesive transdermal therapeutic system such as a patch, or in a form suitable for administration by spraying or by vaporization.

77. The kit according to claim 13, wherein the extract of tobacco leaves or the composition is provided in a dosage form of 0.03 mL to 10 mL.
